(19) United States
(12) Patent Application Publication
(10) Pub. No.: US 2021/0196609 A1
(43) Pub. Date: Jul. 1, 2021

(54) COMPOSITIONS AND METHODS FOR STYLING HAIR

(71) Applicant: L'OREAL, Paris (FR)

(72) Inventors: Aakash Jagat PAREKH, Edison, NJ (US); Siva MUTHUKRISHNAN, Bridgewater, NJ (US); Brady ZARKET, Union, NJ (US); Ronak RUGHANI, Edison, NJ (US)

(21) Appl. No.: 17/130,273

(22) Filed: Dec. 22, 2020

Related U.S. Application Data
(60) Provisional application No. 62/953,869, filed on Dec. 26, 2019.

(51) Int. Cl. A61K 8/60; A61K 8/81; A61K 8/73; A61Q 5/04; A61Q 5/06

(52) U.S. Cl. CPC A61K 8/604; A61K 8/8164; A61Q 5/06; A61K 8/73; A61Q 5/04; A61K 8/733

(57) ABSTRACT

The instant disclosure relates to compositions and methods for styling hair. The compositions comprise at least one polyphenol or a combination of at least one polyphenol and at least one film forming polymer. The methods comprise applying the compositions to hair.

### CROSS-REFERENCE TO RELATED APPLICATIONS

This is a non-provisional patent application which claims priority to U.S. Provisional Patent Application No. 62/953,869, filed on Dec. 26, 2019, which is incorporated by reference herein in its entirety.

### TECHNICAL FIELD

The present disclosure relates to compositions and methods for styling hair.

### BACKGROUND

Consumers desire natural compositions for styling hair. Chemicals and raw materials used in conventional compositions may lack sustainable sourcing and therefore not comply with “green” manufacturing processes, which may make the compositions less desirable to consumers. In addition, consumers wish to avoid the use of conventional styling processes that may be damaging to hair, such as heat-styling hair with heat tools (e.g., flat iron, curling iron) or chemical styling treatments (e.g., perming, relaxing). Further, consumers seek hair styling compositions and methods that permit the hair style to be maintained over an extended period of time and under conditions such as high humidity.

Thus, there is a need for hair styling compositions and methods for styling hair, wherein the compositions have desired cosmetic and composition properties that allow heat-free styling and/or avoid or reduce the amount of synthetic chemicals, yet achieve levels of hair styling similar to that of conventional compositions and methods, and which last over time and in high humidity.

### SUMMARY

It has surprisingly been found that compositions according to the disclosure provide improved hair styling properties, including hair curling and straightening, that is comparable to or better than conventional hair styling formulations and/or to conventional hair styling processes, such as heat-styling the hair with heat tools (e.g., flat iron, curling iron). Additionally, compositions according to the disclosure provide reversible hair styling, in contrast to traditional harsh chemical styling treatments (e.g., perming, relaxing). Hair styled with compositions according to the disclosure maintains its style over an extended period of time and/or under high humidity. Finally, compositions according to the disclosure may, in certain embodiments, comprise greater amounts of materials that are of sustainable and/or natural and/or organic sourcing, and/or reduced amounts of synthetic materials or chemicals. For example, certain embodiments enable reduced amounts of film-forming polymer (e.g., up to ten times less), yet achieve and maintain a hair style as well as or better than a conventional styling composition comprising greater amounts of such polymers.

In one embodiment, the compositions are hair styling compositions that comprise (a) at least one polyphenol, and (b) at least one film forming polymer.

In an embodiment, the hair styling compositions comprise:
(a) at least one polyphenol, and
(b) at least one film forming polymer.

In a further embodiment, the pH of the composition is approximately at or below the pKa of the polyphenol.

In an embodiment, the at least one film forming polymer is present in an amount ranging from about 0.01% to about 10%, about 0.01% to about 5%, about 0.05% to about 3%, or about 0.1% to about 2%, based on the total weight of the composition.

In an embodiment, the at least one film forming polymer is chosen from acrylate-based polymers, polyurethanes, polysaccharides, or mixtures thereof. The at least one film forming polymer. Non-limiting examples include PVM/MA Copolymer, carrageenan, gum Arabic, xanthan gum, alginate, or mixtures thereof.

In an embodiment, the at least one polyphenol is present in an amount ranging from about 0.001% to about 20%, about 0.01% to about 15%, or about 0.1% to about 10%, based on the total weight of the composition.

In an embodiment, the at least one polyphenol is tannic acid.

The pH of the composition may be less than about 8.

In a further embodiment, the composition may comprise from about 50% to about 99% solvent by weight relative to the total composition. The composition may comprise from about 50% to about 99% water by weight relative to the total composition.

In an embodiment, the weight ratio of the total amount of the polyphenol to the total amount of film forming polymer may range from about 30:1 to about 1:30, from about 30:1 to about 1:1, from about 10:1 to about 1:1, from about 5:1 to about 1:1, or from about 5:1 to about 10:1. In an embodiment, the weight ratio of the total amount of the tannic acid to the total amount of film forming polymer ranges from about 5:1 to about 1:1.

In a further embodiment, the hair styling composition is substantially free of synthetic gums, silicones, or synthetic polymers.

The methods for styling hair may comprise:
(1) applying to the hair a hair styling composition comprising:
   (a) at least one polyphenol, and
   (b) at least one film forming polymer; and
(2) styling the hair.

In an embodiment, the pH of the composition is approximately at or below the pKa of the polyphenol. In one embodiment, the hair styling composition is applied to wet hair and the hair is dried after the hair is styled.

Styling the hair may comprise straightening the hair or curling the hair. In a further embodiment, styling the hair may comprise elongating the curl of the hair.

In an embodiment, the hair styling composition may comprise
(a) tannic acid,
(b) at least one film forming polymer chosen from PVM/MA
Copolymer, carrageenan, gum Arabic, xanthan gum, alginate, or mixtures thereof, and
(c) at least one solvent,
wherein the pH of the composition is at or below 6.

The hair styling composition may comprise:
(a) tannic acid, and
(b) at least one film forming polymer of formula (I):

[CH₂CH(OCH₃)CH(CO₂H)CH(CO₂H)]n,
wherein n is a number from about 2,500 to about 17,500, from about 3,000 to about 17,000, from about 3,500 to about 16,500, from about 4,000 to about 16,000, from about 4,500 to about 15,500, from about 5,000 to about 15,000, from about 5,500 to about 14,5000, from about 6,000 to about 14,000, from about 6,500 to about 7,000, from about 6,500 to about 13,500, from about 7,000 to about 13,000, from about 7,500 to about 12,500, from about 8,000 to about 12,000, or from about 8,500 to about 11,500.

The hair styling composition may comprise:
(a) tannic acid, and
(b) carrageenan.

The hair styling composition may comprise:
(a) tannic acid, and
(b) alginate.

The hair styling composition may comprise:
(a) tannic acid, and
(b) xanthan gum.

The hair styling composition may comprise:
(a) tannic acid, and
(b) gum Arabic.

In an embodiment, the method for styling hair may comprise:
(1) applying to the hair a hair styling composition comprising:
   (a) tannic acid,
   (b) at least one film forming polymer chosen from PVM/MA Copolymer, carrageenan, gum Arabic, xanthan gum, alginate, or mixtures thereof, and
   (c) at least one solvent,
      wherein the pH of the composition is at or below 6; and
(2) styling the hair.

In an embodiment, the method for styling hair may comprise:
(1) applying to the hair a hair styling composition comprising tannic acid, and
(2) styling the hair.

The composition for use in the method for styling hair may comprise:
(a) tannic acid, and
(b) at least one film forming polymer of formula (I):

[CH₂CH(OCH₃)CH(CO₂H)CH(CO₂H)]n,
wherein n is a number from about 2,500 to about 17,500, from about 3,000 to about 17,000, from about 3,500 to about 16,500, from about 4,000 to about 16,000, from about 4,500 to about 15,500, from about 5,000 to about 15,000, from about 5,500 to about 14,500, from about 6,000 to about 14,000, from about 6,500 to about 13,500, from about 7,000 to about 13,000, from about 7,500 to about 12,500, from about 8,000 to about 12,000, from about 8,500 to about 11,500, from about 9,000 to about 11,000, or from about 9,500 to about 10,500.

In an embodiment, the composition for use in the method for styling hair may comprise:
(a) tannic acid, and
(b) carrageenan.

In an embodiment, the composition for use in the method for styling hair may comprise:
(a) tannic acid, and
(b) alginate.

In an embodiment, the composition for use in the method for styling hair may comprise:
(a) tannic acid, and
(b) xanthan gum.

In an embodiment, the composition for use in the method for styling hair may comprise:
(a) tannic acid, and
(b) gum Arabic.

In an embodiment, the disclosed hair styling compositions and methods comprise at least 85% of the materials used in the composition are plant-based or of plant origin.

A hair styling composition comprising tannic acid is also disclosed herein. In an embodiment, the hair styling composition further comprises at least one film forming polymer. In an embodiment, the pH of the composition is approximately at or below the pKa of the polyphenol.

In an embodiment, the tannic acid is present in an amount ranging from about 0.001% to about 20%, or about 4% to about 6%, based on the total weight of the composition. In an embodiment, the pH of the composition is less than about 8.

In a further embodiment, the at least one film forming polymer is present in an amount ranging from about 0.01% to about 10%, or about 0.05% to about 3%, based on the total weight of the composition. The film forming polymer may, for example, be chosen from acrylate-based polymers, polyurethanes, polysaccharides, or mixtures thereof. Non-limiting examples of the at least one film forming polymer is chosen from PVM/MA Copolymer, carrageenan, gum Arabic, xanthan gum, alginate, or mixtures thereof. In an embodiment, the weight ratio of the total amount of the tannic acid to the total amount of film forming polymer ranges from about 1:30 to about 30:1, or about 5:1 to about 10:1.

A method of styling composition comprising applying to the hair a hair styling composition comprising tannic acid is also disclosed herein. The method may comprise, for example, applying the composition to wet hair wherein the hair is dried after the hair is styled, and/or styling the hair comprises straightening the hair, curling the hair, or comprises elongating the curl of the hair.

### BRIEF DESCRIPTION OF THE FIGURES

FIGS. 1A-1B show photographs of swatches of hair treated with compositions comprising citric acid, tannic acid, and/or Gantrez® PVM/MA at time=0 (FIG. 1A) and after 24 hours at 80% relative humidity and 25° C. (FIG. 1B).

FIGS. 2A-2B show photographs of swatches of hair treated with compositions comprising tannic acid and/or Gantrez® PVM/MA at time=0 (FIG. 2A) and after 24 hours at 80% relative humidity and 25° C. (FIG. 2B).

FIGS. 3A-3B show photographs of swatches of hair treated with compositions comprising tannic acid and/or carrageenan at time=0 (FIG. 3A) and after 24 hours at 80% relative humidity and 25° C. (FIG. 3B).

FIGS. 4A-4B show photographs of swatches of hair treated with compositions comprising tannic acid and/or alginate at time=0 (FIG. 4A) and after 24 hours at 80% relative humidity and 25° C. (FIG. 4B).

FIGS. 5A-5B show photographs of swatches of hair treated with compositions comprising tannic acid and/or xanthan gum at time=0 (FIG. 5A) and after 24 hours at 80% relative humidity and 25° C. (FIG. 5B).

FIGS. 6A-6B show photographs of swatches of hair treated with compositions comprising tannic acid and/or gum Arabic at time=0 (FIG. 6A) and after 24 hours at 80% relative humidity and 25° C. (FIG. 6B).

FIGS. 7A-7B show photographs of swatches of hair treated with compositions comprising tannic acid at time=0 (FIG. 7A) and after 24 hours at 80% relative humidity and 25° C. (FIG. 7B).

FIGS. 8A-8B show photographs of swatches of hair treated with compositions comprising water at time=0 (FIG. 8A) and after 24 hours at 80% relative humidity and 25° C. (FIG. 8B).

FIG. 9 shows photographs of swatches of hair treated with compositions comprising water, glycerol, citric acid, tannic acid, Gantrez® PVM/MA and/or carrageenan at time=0 and after 24 hours at ambient temperature.

FIG. 10 shows photographs of swatches of hair treated with compositions comprising water, glycerol, citric acid, tannic acid, Gantrez® PVM/MA and/or carrageenan at time=0, 1 hour and after 24 hours at 80% relative humidity and 25° C.

FIG. 11 shows graphs of global dispersion (degree of curliness) for swatches of hair treated with the treatment (0.5% Carrageenan+5% Tannic Acid q.s. in DI water) and control (water) along with the corresponding photographs of treated swatches and control swatches taken after air drying.

FIG. 12 shows graphs of the average fiber alignment coefficient (degree of straight alignment) for swatches of hair treated with the treatment (0.5% Carrageenan+5% Tannic Acid q.s. in DI water) and control (DI water) along with the corresponding photographs of treated swatches and control swatches taken after air drying.

FIG. 13 shows photographic images of the hair of a human volunteer treated on one side with a composition comprising 0.5% Carrageenan+5% Tannic Acid q.s. in DI water (treatment) and on the other side with a control (DI water).

### DESCRIPTION

The disclosure relates to compositions and methods for styling hair. The compositions comprise polyphenols, or synergistic combinations of polyphenols and polymers. The methods comprise applying the compositions to the hair and styling the hair.

### I. Compositions

The compositions according to the disclosure comprise polyphenols or unexpectedly synergistic combinations of polyphenols and polymers, which surprisingly lead to improved hair styling properties.

Polyphenols

Compositions according to the disclosure comprise at least one polyphenol. Polyphenols are phenols with more than one phenolic —OH group that have the ability to act as “donor molecules” by donating their alcoholic hydrogen or accepting delocalized electrons. The two classes of polyphenols are flavonoids and non-flavonoids.

Exemplary and non-limiting flavonoid compounds that can be used include: chalcones, such as phloretin, phloridzin, aspalathin, or neohesperidine; flavanols, such as catechin, fisetin, kaempferol, myricetin, quercetin, rutin, proanthocyanidins, pyroanthocyanidins, theaflavins, or thearubigins (or thearubrins); dihydroflavonols, such as astilbin, dihydroquercetin, or silibinin; flavanones, such as hesperidin, neohesperidin, hesperetin, naringenin, naringin, or poncirin; flavones, such as apigenin, baicalin, diosmin, or rhoifolin; anthocyanins, such as cyanidin, delphinidin, malvidin, peonidin, or petunidin; tannins, such as ellagitannins, tannic acid, gallic acid, or ellagic acid; isoflavonoids, such as biochanin A, Daidzein, or Genistein; fulvic acid, and neoflavanoids, as well as combinations thereof.

Exemplary and non-limiting non-flavonoid compounds that can be used include: Curcuminoids such as curcumin or tetrahydrocurcumin; Stibenoids such as astringin, resveratrol, or rhaponticin; Aurones such as bracteatin, or aureusidin; and Lignans such as pinoresinol, as well as combinations thereof.

Other polyphenols that can be used include hydroxycinnamic acids, for example, chlorogenic acid, verbascoside; phenolic aldehydes; phenylpropenes; coumarins, coumestans, or tyrosols, as well as combinations thereof. In one embodiment, the polyphenols may be plant-based and/or organic.

In certain exemplary embodiments, polyphenols useful according to the disclosure may be chosen from tannic acid, resveratrol, catechin, ellagic acid, resorcinol, gallic acid, humic acid, chlorogenic acid, quercetin, anthocyanin, chebulinic acid or mixtures thereof. In one exemplary embodiment, the polyphenol is tannic acid.

The total amount of the at least one polyphenol may vary, but typically ranges from about 0.001% to about 20%, about 0.01% to about 15%, or about 0.1% to about 10%, based on the total weight of the composition. For example, the total amount of the at least polyphenol may range from about 0.01% to about 10%, such as from about 0.01% to about 9%, about 0.01% to about 8%, about 0.01% to about 7%, about 0.01% to about 6%, about 0.01% to about 5%, about 0.01% to about 4%, about 0.01% to about 3%, about 0.01% to about 2%, about 0.01% to about 1%, about 0.01% to about 0.75%, about 0.01% to about 0.5%, about 0.05% to about 10%, about 0.05% to about 9%, about 0.05% to about 8%, about 0.05% to about 7%, about 0.05% to about 6%, about 0.05% to about 5%, about 0.05% to about 4%, about 0.05% to about 3%, about 0.05% to about 2%, about 0.05% to about 1%, about 0.05% to about 0.75%, about 0.05% to about 0.5%, about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1%, about 0.1% to about 0.75%, about 0.1% to about 0.5%, about 0.5% to about 10%, about 0.5% to about 9%, about 0.5% to about 8%, about 0.5% to about 7%, about 0.5% to about 6%, about 0.5% to about 5%, about 0.5% to about 4%, about 0.5% to about 3%, about 0.5% to about 2%, about 0.5% to about 1%, about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 10%, about 2% to about 9%, about 2% to about 8%, about 2% to about 7%, about 2% to about 6%, about 2% to about 5%, about 2% to about 4%, about 2% to about 3%, about 3% to about 10%, about 3% to about 9%, about 3% to about 8%, about 3% to about 7%, about 3% to about 6%, about 3% to about 5%, about 3% to about 4%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, or about 5% to about 6%, including all ranges and sub-ranges there between, based on the total weight of the composition. In various embodiments, the at least one polyphenol may be present in an amount of about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10%, based on the total weight of the composition. In certain embodiments, the polyphenol is tannic acid.

Polymers

The compositions described herein optionally comprise at least one film forming or fixing polymer. As used herein, the terms “film forming polymer” or “fixing polymer” are understood to mean any polymer that is capable, when applied to the hair, of forming a film on hair, thereby allowing the hair to achieve and/or retain a desired shape. Without wishing to be bound by theory, it is believed that the polyphenol may act as a crosslinker to the polymer, which unexpectedly results in synergistically improved hair styling properties.

Useful film forming polymers include synthetic, semi-synthetic, or natural and/or plant-based and/or organic polymers. Any anionic, cationic, amphoteric, and non-ionic film forming polymers, as well as mixtures thereof, may be used in the disclosed compositions and methods.

By way of non-limiting example, anionic film forming polymers that can be used in the disclosed compositions are polymers comprising groups derived from carboxylic acid, sulfonic acid or phosphoric acid and have a number-average molecular weight of ranging from approximately 500 and 5,000,000.

The carboxylic groups are provided by unsaturated monocarboxylic or dicarboxylic acid monomers such as those corresponding to the formula (II):

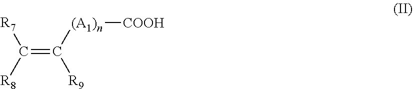

wherein n is an integer from 0 to 10, A₁ denotes a methylene group optionally joined to the carbon atom of the unsaturated group or to the adjacent methylene group when n is greater than 1, via a heteroatom such as oxygen or sulfur, R₇ denotes a hydrogen atom or a phenyl or benzyl group, R₈ denotes a hydrogen atom or a lower alkyl or carboxyl group, and R₉ denotes a hydrogen atom, a lower alkyl group, or a CH₂—COOH, phenyl or benzyl group.

In the abovementioned formula, a lower alkyl group may denote a group having 1 to 4 carbon atoms, and in particular the methyl and ethyl groups.

By way of non-limiting example, anionic film forming polymers comprising carboxylic groups which may be used in the disclosed compositions are:

A) copolymers of acrylic or methacrylic acid or salts thereof. Among these polymers, mention may be made of copolymers of acrylic or methacrylic acid with a monoethylenic monomer such as ethylene, styrene, vinyl esters, acrylic or methacrylic acid esters, optionally grafted onto a polyalkylene glycol such as polyethylene glycol and optionally crosslinked. Such polymers are described in particular in French patent No.1 222 944 and German patent application No. 2 330 956, the copolymers of this type comprising an optionally N-alkylated and/or hydroxyalkylated acrylamide unit in their chain as described in particular in Luxembourg patent application Nos. 75370 and 75371. Mention may also be made of copolymers of acrylic acid and of C₁-C₄ alkyl methacrylate and terpolymers of vinylpyrrolidone, of acrylic acid and of C₁-C₂₀ alkyl methacrylate, for example lauryl methacrylate, such as the product sold by the company ISP under the name Acrylidone® LM (INCI name VP/acrylates/lauryl methacrylate copolymer), acrylic acid/ethyl acrylate/N-t-butylacrylamide terpolymers, such as the products Ultrahold® Strong and Ultrahold® 8 sold by the company BASF (INCI name Acrylates/t-butylacrylamide copolymer), methacrylic acid/ethyl acrylate/tert-butyl acrylate terpolymers, such as the products sold under the names Luvimer® 100 P or Luvimer® PRO 55 by the company BASF (INCI name Acrylates copolymer), copolymers of methacrylic acid and of ethyl acrylate, such as the products sold under the names Luvimer® MAE or Luviflex® Soft by the company BASF (INCI name Acrylates copolymer), acrylic acid/butyl acrylate/methyl methacrylate terpolymers, such as the product sold under the name Balance® CR by the company Akzo Nobel (INCI name Acrylates copolymer), and the copolymers of methacrylic acid and of methyl methacrylate sold under the name Eudragit® L 100 by the company Rohm Pharma (I NCI name Acrylates copolymer). Mention may also be made of branched block polymers containing (meth)acrylic acid monomers, such as the product sold under the name Fixate® G-100L by the company Lubrizol (INCI name AMP-acrylates/allyl methacrylate copolymer);
B) crotonic acid copolymers, such as those comprising vinyl acetate or propionate units in their chain and optionally other monomers such as allyl esters or methallyl esters, vinyl ether or vinyl ester of a linear or branched saturated carboxylic acid with a long hydrocarbon-based chain, such as those containing at least 5 carbon atoms, it being possible for these polymers optionally to be grafted or crosslinked, or alternatively another vinyl, allyl or methallyl ester monomer of an α- or β-cyclic carboxylic acid. Such polymers are described, inter alia, in French patent Nos. 1 222 944, 1 580 545, 2 265 782, 2 265 781, 1 564 110 and 2 439 798. Commercial products which fall into this category are the products Resyn® 28-2930 and 28-1310 sold by the company Akzo Nobel (INCI names VA/crotonates/vinyl decanoate copolymer and VA/crotonates copolymer, respectively). Mention may also be made of the products Luviset® CA 66 sold by the company BASF, Aristoflex® A60 sold by the company Clariant (INCI name VA/crotonates copolymer) and Mexomere® PW or PAM sold by the company Chimex (INCI name VA/vinyl butyl benzoate/crotonates copolymer);

C) copolymers of monounsaturated C₄-C₈ carboxylic acids or anhydrides selected from:
copolymers comprising (i) one or more maleic, fumaric or itaconic acids or anhydrides and (ii) at least one monomer chosen from vinyl esters, vinyl ethers, vinyl halides, phenylvinyl derivatives, acrylic acid and its esters, the anhydride functions of these copolymers optionally being monoesterified or monoamidated. Such polymers are described, in particular, in U.S. Pat. Nos. 2,047,398, 2,723,248 and 2,102,113, and GB patent No. 839 805. By way of non-limiting example, maleic acid/anhydride copolymers that may be useful include methyl vinyl ether-maleic acid copolymer, such as that available commercially from ISP under the tradename GANTREZ® S (e.g., GANTREZ® S-97 BF; INCI name PVM/MA copolymer), methyl vinyl ether-maleic anhydride copolymer, such as that available commercially from ISP under the tradename GANTREZ® AN (INCI name polymethylvinylether/maleic anhydride copolymer), isobutyl vinyl ether-maleic anhydride copolymer, ethylene-maleic anhydride copolymer, isobutylene-maleic anhydride copolymer, styrene-maleic anhydride copolymer, vinyltriazole-maleic anhydride copolymer and vinylnorbornene-maleic anhydride copolymer. The copolymers may contain, in addition to the at least one copolymerizable monomer, both maleic acid and maleic anhydride, or a derivative thereof. Additional useful commercial products include those sold under the names Gantrez® ES by the company ISP, such as Gantrez® ES 225 (INCI name ethyl ester of PVM/MA copolymer) or Gantrez® ES 425L (INCI name butyl ester of PVM/MA copolymer). In an embodiment, the disclosed polymer may be Gantrez® PVM/MA (Poly(methyl vinyl ether-alt-maleic acid)(CAS Number 25153-40-6). The PVM/MA copolymer, or Poly(methyl vinyl ether-alt-maleic acid, may have an average Mw of approximately 1,980,000 by LS, and/or an average Mn of approximately 960,000 (powder). For example, the molecular weight of PVM/MA may be from about 75,000 to about 3,000,000 g/mol, about 150,000 to about 2,750,000 g/mol, about 300,000 to about 2,500,000 g/mol, about 450,000 to about 2,250,000 g/mol, about 600,000 to 2,200,000 about g/mol, about 850,000 to 2,150,000 about g/mol, about 1,100,000 to 2,100,000 about g/mol, about 1,225,000 to 2,005,000 about g/mol, about 1,500,000 and about 2,000,000 g/mol about 1,650,000 to 1,850,000 about g/mo. The PVM/MA (Poly(methyl vinyl ether-alt-maleic acid) may have a formula of formula (I):

[CH₂CH(OCH₃)CH(CO₂H)CH(CO₂H)]n (I)

wherein n is a number from about 2,500 to about 17,500, from about 3,000 to about 17,000, from about 3,500 to about 16,500, from about 4,000 to about 16,000, from about 4,500 to about 15,500, from about 5,000 to about 15,000, from about 5,500 to about 14,500, from about 6,000 to about 14,000, from about 6,500 to about 13,500, from about 7,000 to about 13,000, from about 7,500 to about 12,500, from about 8,000 to about 12,000, from about 8,500 to about 11,500, from about 9,000 to about 11,000, or from about 9,500 to about 10,500.
copolymers comprising (i) one or more maleic, citraconic or itaconic anhydride units and (ii) one or more monomers chosen from allyl or methallyl esters optionally comprising one or more acrylamide, methacrylamide, α-olefin, acrylic or methacrylic ester, acrylic or methacrylic acid or vinylpyrrolidone groups in their chain, the anhydride functions of these copolymers optionally being monoesterified or monoamidated. These polymers are described, for example, in French patent Nos. 2 350 384 and 2 357 241;
D) polyacrylamides comprising carboxylate groups. The film forming polymers comprising units derived from sulfonic acid can be chosen from:

A′) homopolymers and copolymers comprising vinylsulfonic, styrenesulfonic, naphthalenesulfonic or acrylamidoalkylsulfonic units.

These polymers can be chosen in particular from:
polyvinylsulfonic acid salts having a molecular weight of approximately ranging from 1000 and 100,000, and also the copolymers with an unsaturated comonomer such as acrylic or methacrylic acids and their esters, and also acrylamide or its derivatives, vinyl ethers and vinylpyrrolidone;
polystyrenesulfonic acid salts such as the sodium salts that are sold for example under the name Flexan® II by Akzo Nobel (INCI name Sodium polystyrene sulfonate). These compounds are described in patent FR 2 198 719;
polyacrylamidosulfonic acid salts, such as those mentioned in U.S. Pat. No. 4,128,631, and more particularly the polyacrylamidoethylpropanesulfonic acid, sold under the name Rheocare® HSP-1180 by Cognis (INCI name polyacrylamidomethyl-propane sulfonic acid);

B′) sulfonic polyesters, these polymers being obtained by polycondensation of at least one dicarboxylic acid, of at least one diol or of a mixture of diol and of diamine, and of at least one difunctional monomer comprising a sulfonic function. Among these polymers, mention may be made of:
linear sulfonic polyesters such as those described in U.S. Pat. Nos. 3,734,874, 3,779,993, 4,119,680, 4,300,580, 4,973,656, 5,660,816, 5,662,893 and 5,674,479. Such polymers are, for example, the products Eastman® AQ38S Polymer, Eastman® AQ55S Polymer and Eastman® AQ48 Ultra Polymer sold by the company Eastman Chemical (name Polyester-5) which are copolymers obtained from diethylene glycol, from 1,4-cyclohexanedimethanol, from isophthalic acid and from sulfoisophthalic acid salt;
branched sulfonic polyesters such as those described in patent applications WO 95/18191, WO 97/08261 and WO 97/20899. Such compounds are, for example, the products Eastman® AQ10D Polymer (name Polyester-13) or Eastman® AQ1350 Polymer provided by the company Eastman Chemical (name Polyester-13).

In an embodiment, the anionic film forming polymers are chosen from copolymers of acrylic acid, such as the acrylic acid/ethyl acrylate/N-tert-butylacrylamide terpolymers sold in particular under the name Ultrahold® Strong by the company BASF, copolymers derived from crotonic acid, such as vinyl acetate/vinyl tert-butylbenzoate/crotonic acid terpolymers and the crotonic acid/vinyl acetate/vinyl neododecanoate terpolymers sold in particular under the name Resyn 28-2930 by the company Akzo Nobel, polymers derived from maleic, fumaric or itaconic acids or anhydrides with vinyl esters, vinyl ethers, vinyl halides, phenylvinyl derivatives and acrylic acid and esters thereof, such as the methyl vinyl ether/monoesterified maleic anhydride copolymers sold, for example, under the names Gantrez® ES 425L or ES 225 by the company ISP, the copolymers of methacrylic acid and of ethyl acrylate sold under the name Luvimer® MAE by the company BASF, and the vinyl acetate/crotonic acid copolymers sold under the name Luviset® CA 66 by the company BASF, and the vinyl acetate/crotonic acid copolymers grafted with polyethylene glycol sold under the name Aristoflex® A60 by the company Clariant, the vinylpyrrolidone/acrylic acid/lauryl methacrylate terpolymers sold under the name Acrylidone® LM by the company ISP, the polymer sold under the name Fixate® G-100L by the company Lubrizol, the vinyl acetate/crotonic acid/vinyl p-tert-butylbenzoate copolymers sold under the names Mexomere® PW or PAM by the company Chimex.

By way of non-limiting example, the cationic film forming film-forming polymers that can be used in the disclosed composition may be chosen from polymers comprising primary, secondary, tertiary and/or quaternary amine groups forming part of the polymer chain or directly attached thereto, and having a molecular weight of ranging from 500 and about 5,000,000 and preferably ranging from 1000 and 3,000,000.

Among these polymers, mention may be made more particularly of the following cationic polymers:

(1) homopolymers or copolymers derived from acrylic or methacrylic esters or amides and comprising at least one of the units of the following formulae:

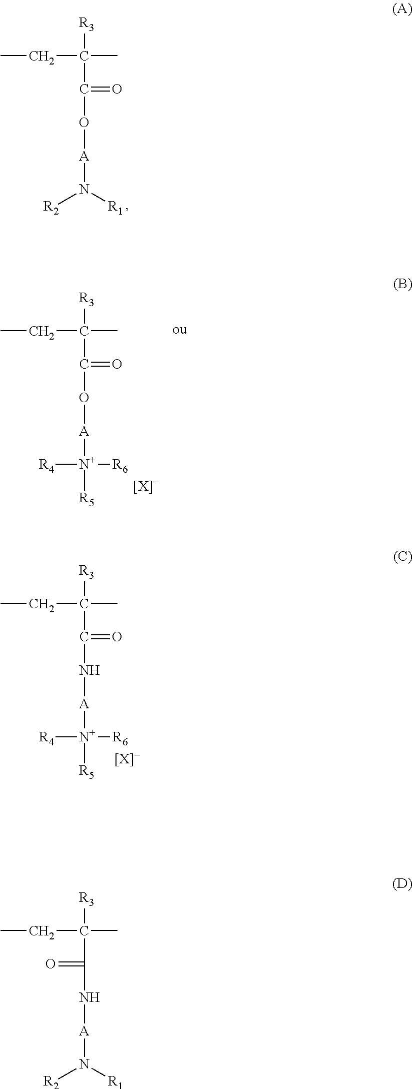

in which:
R₃ denotes a hydrogen atom or a CHs radical;
A is a linear or branched alkyl group comprising from 1 to 6 carbon atoms or a hydroxyalkyl group comprising from 1 to 4 carbon atoms;
R₄, R₅ and R₆, which may be identical or different, represent an alkyl group having from 1 to 18 carbon atoms or a benzyl radical;
R₁ and R₂, which may be identical or different, each represent a hydrogen atom or an alkyl group having from 1 to 6 carbon atoms;
X denotes a methosulfate anion or a halide such as chloride or bromide.

The copolymers of family (1) also contain one or more units derived from comonomers that may be chosen from the family of acrylamides, methacrylamides, diacetone acrylamides, acrylamides and methacrylamides substituted on the nitrogen with lower (C₁-C₄) alkyl groups, groups derived from acrylic or methacrylic acids or esters thereof, vinyllactams such as vinylpyrrolidone or vinylcaprolactam, and vinyl esters.

Thus, among these copolymers of family (1), mention may be made of:
quaternized or non-quaternized vinylpyrrolidone/dialkylaminoalkyl acrylate or methacrylate copolymers, such as the products sold under the name Gafquat® by the company ISP, for instance Gafquat® 734 or Gafquat® 755 or Gafquat® 755N (INCI name Polyquaternium-11), or alternatively the products known as Copolymer® 845, 958 and 937 sold by ISP (INCI name VP/dimethylaminoethyl methacrylate copolymer). These polymers are described in detail in French patents 2 077 143 and 2 393 573,
fatty-chain polymers containing a vinylpyrrolidone unit, such as the products sold under the name Styleze® W20L and Styleze® W10 by the company ISP (INCI name Polyquaternium-55),
dimethylaminoethyl methacrylate/vinylcaprolactam/vinyl pyrrolidone terpolymers, such as the products sold under the names Advantage HC 37 or Gaffix® VC 713 by the company ISP (INCI name Vinyl caprolactam/VP/dimethylaminoethyl methacrylate copolymer), and
quaternized vinylpyrrolidone/dimethylaminopropylmethacrylamide copolymers, such as the products sold under the name Gafquat® HS 100 by the company ISP (name Polyquaternium-28);

(2) cationic guar gum derivatives, preferably containing quaternary ammonium, such as those described in U.S. Pat. Nos. 3,589,578 and 4,031,307, such as guar gums containing trialkylammonium cationic groups. Such products are sold in particular under the trade names Jaguar® C13 S, Jaguar® C 15 and Jaguar® C 17 by the company Rhodia (INCI name Guar hydroxypropyltrimonium chloride);

(3) quaternary copolymers of vinylpyrrolidone and of vinylimidazole; mention may be made, for example, of vinylpyrrolidone/methylvinylimidazolium chloride copolymers, such as the products sold by the company BASF under the names Luviquat® FC550 or FC370, Luviquat® Excellence and Luviquat® Style (INCI name Polyquaternium-16), or vinylpyrrolidone/vinylimidazolium methosulfate/vinylcaprolactam terpolymers, such as the product Luviquat® Hold sold by the company BASF (INCI name Polyquaternium-46);

(4) chitosans or salts thereof; the salts that can be used are, in particular, chitosan acetate, lactate, glutamate, gluconate or pyrrolidonecarboxylate. Among these compounds, mention may be made of the chitosan pyrrolidonecarboxylate sold under the name Kytamer® PC by the company Amerchol (INCI name Chitosan PCA);

(5) cationic cellulose derivatives such as copolymers of cellulose or of cellulose derivatives grafted with a water-soluble monomer comprising a quaternary ammonium, and described in particular in U.S. Pat. No. 4,131,576, such as hydroxyalkylcelluloses, for instance hydroxymethyl-, hydroxyethyl- or hydroxypropylcelluloses grafted in particular with a methacryloyloxyethyltrimethylammonium, methacrylamidopropyltrimethyl-ammonium or dimethyldiallylammonium salt. The commercial products corresponding to this definition are, more particularly, the products sold under the name Celquat® L 200 and Celquat® H 100 by the company Akzo Nobel (INCI name Polyquaternium-4).

The amphoteric film forming polymers that may be used in accordance with the invention may be selected from polymers comprising units B and C distributed statistically in the polymer chain, where B denotes a unit derived from a monomer comprising at least one basic nitrogen atom and C denotes a unit derived from an acid monomer comprising one or more carboxylic or sulfonic groups, or alternatively B and C may denote groups derived from carboxybetaine or sulfobetaine zwitterionic monomers.;

Additionally, B and C can also denote a cationic polymer chain comprising primary, secondary, tertiary or quaternary amine groups, in which at least one of the amine groups bears a carboxylic or sulfonic group connected via a hydrocarbon-based group, or alternatively B and C form part of a chain of a polymer comprising an α,β-dicarboxylic ethylene unit in which one of the carboxylic groups has been made to react with a polyamine comprising one or more primary or secondary amine groups.

The amphoteric film forming polymers corresponding to the definition given above may be chosen from the following polymers:

(1) copolymers containing acidic vinyl units and basic vinyl units, such as those resulting from the copolymerization of a monomer derived from a vinyl compound bearing a carboxylic group such as, more particularly, acrylic acid, methacrylic acid, maleic acid, α-chloroacrylic acid, and of a basic monomer derived from a substituted vinyl compound containing at least one basic atom, such as, more particularly, dialkylaminoalkyl methacrylate and acrylate, dialkylaminoalkylmethacrylamide and acrylamide. Such compounds are described in U.S. Pat. No. 3,836,537;

(2) polymers comprising units deriving from:
a) at least one monomer chosen from acrylamides or methacrylamides substituted on the nitrogen atom with an alkyl group,
b) at least one acidic comonomer containing one or more reactive carboxylic groups, and
c) at least one basic comonomer such as esters containing primary, secondary, tertiary and quaternary amine substituents of acrylic and methacrylic acids and the product of quaternization of dimethylaminoethyl methacrylate with dimethyl or diethyl sulfate.

The N-substituted acrylamides or methacrylamides may be chosen from compounds in which the alkyl groups contain from 2 to 12 carbon atoms and more particularly N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers may be chosen from acrylic, methacrylic, crotonic, itaconic, maleic and fumaric acid and also alkyl monoesters, having 1 to 4 carbon atoms, of maleic or fumaric acid or anhydride. The basic comonomers may be chosen from aminoethyl, butylaminoethyl, N,N′-dimethylaminoethyl and N-tert-butylaminoethyl methacrylates.

The copolymers of which the INCI name is octylacrylamide/acrylates/butylaminoethyl methacrylate copolymer, such as the products sold under the names Amphomer®, Amphomer® LV71 or Balance® 47 by the company Akzo Nobel, may be used;

(3) crosslinked and acylated polyaminoamides partially or totally deriving from polyaminoamides of general formula (III):

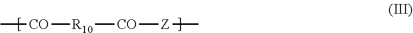

in which:
R₁₀ represents a divalent group derived from a saturated dicarboxylic acid, a mono- or dicarboxylic aliphatic acid containing an ethylenic double bond, an ester of a lower alkanol having 1 to 6 carbon atoms of these acids, or a group derived from the addition of any one of said acids to a bis(primary) or bis(secondary) amine, and
Z denotes a group derived from a bis(primary), mono(secondary) or bis(secondary) polyalkylene-polyamine and may represent:
   a) in proportions of from 60 to 100 mol %, the group of formula:

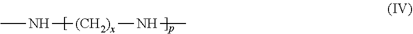

where x=2 and p=2 or 3, or alternatively x=3 and p=2,
this group deriving from diethylenetriamine, from triethylenetetramine or from dipropylenetriamine;
b) in proportions of from 0 to 40 mol %, the group (IV) above in which x=2 and p=1 and which is derived from ethylenediamine, or the group deriving from piperazine for formula (V):

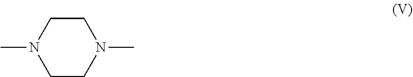

c) in proportions of from 0 to 20 mol %, the group —NH—(CH₂)₆—NH— deriving from hexamethylenediamine,

these polyaminoamides being crosslinked by addition reaction of a difunctional crosslinking agent chosen from epihalohydrins, diepoxides, dianhydrides and bis-unsaturated derivatives, using from 0.025 to 0.35 mol of crosslinking agent per amine group of the polyaminoamide and acylated by the action of acrylic acid, chloroacetic acid or an alkane sultone, or salts thereof.

The saturated carboxylic acids are preferably chosen from acids having 6 to 10 carbon atoms, such as adipic acid, 2,2,4-trimethyladipic acid and 2,4,4-trimethyladipic acid, terephthalic acid, acids containing an ethylenic double bond such as, for example, acrylic acid, methacrylic acid and itaconic acid.

The alkane sultones used in the acylation may be propane sultone or butane sultone; the salts of the acylating agents may be the sodium or potassium salts;

(4) polymers comprising zwitterionic units of formula (VI):

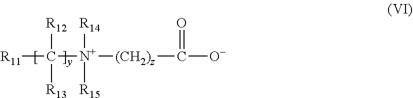

in which:
R₁₁ denotes a polymerizable unsaturated group such as an acrylate, methacrylate, acrylamide or methacrylamide group,
y and z represent an integer from 1 to 3,
R₁₂ and R₁₃ represent a hydrogen atom, or a methyl, ethyl or propyl group, and
R₁₄ and R₁₅ represent a hydrogen atom or an alkyl group such that the sum of the carbon atoms in R₁₄ and R₁₅ does not exceed 10.

The polymers comprising such units may also comprise units derived from non-zwitterionic monomers such as dimethyl-or diethylaminoethyl acrylate or methacrylate or alkyl acrylates or methacrylates, acrylamides or methacrylamides or vinyl acetate. Mention may be made, by way of example, of methyl methacrylate/methyl dimethylcarboxymethylammonioethyl methacrylate copolymers, such as the product sold under the name Diaformer Z-301N or Z-301W by the company Clariant (INCI name Acrylates copolymer);

(5) polymers derived from chitosan comprising monomer units corresponding to the following formula:

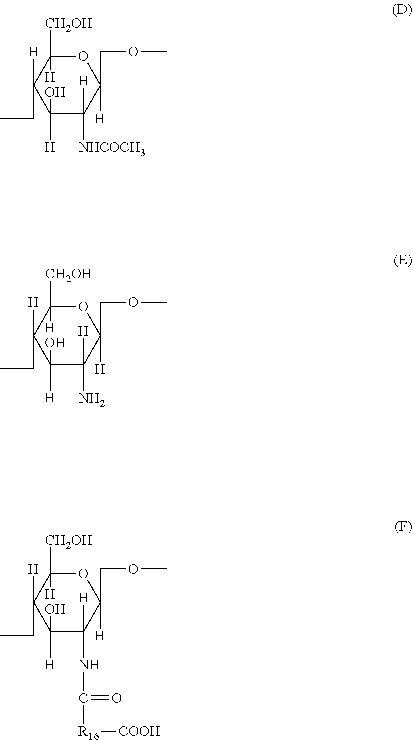

wherein:
the unit (D) is present in proportions ranging from 0% to 30%,
the unit (E) is present in proportions ranging from 5% to 50%, and
the unit (F) is present in proportions ranging from 30% to 90%, it being understood that, in this unit (F), R₁₆ represents a group of formula (VII):

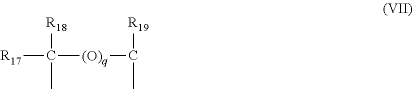

in which,
if q=0, then R₁₇, R₁₈ and R₁₉, which may be identical or different, each represent a hydrogen atom, a methyl, hydroxyl, acetoxy or amino residue, a monoalkylamine residue or a dialkylamine residue that are optionally interspersed with one or more nitrogen atoms and/or optionally substituted with one or more amine, hydroxyl, carboxyl, alkylthio or sulfonic groups, or an alkylthio residue in which the alkyl group bears an amino residue, at least one of the groups R₁₇, R₁₈ and R₁₉ being, in this case, a hydrogen atom; or,
if q=1, then R₁₇, R₁₈ and R₁₉ each represent a hydrogen atom, and also the salts formed by these compounds with bases or acids;

(6) polymers containing units corresponding to general formula (VIII) are described, for example, in French patent 1 400 366:

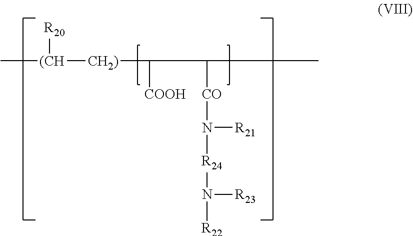

in which:
R₂₀ represents a hydrogen atom, a CH₃O, CH₃CH₂O or phenyl group,
R₂₁ denotes a hydrogen atom or a lower alkyl group such as methyl or ethyl,
R₂₂ denotes a hydrogen atom or a C₁-C₆ lower alkyl group such as methyl or ethyl,
R₂₄ representing a group —CH₂—CH₂—, —CH₂—CH₂—CH₂— or —CH₂—CH(CH₃)—, and
R₂₃ denotes a C₁-C₆ lower alkyl group such as methyl or ethyl or a group corresponding to the formula (IX):

—R₂₄—N(R₂₂)₂ (IX)
with R₂₂ and R₂₄ having the meanings mentioned above;

(7) polymers derived from the N-carboxyalkylation of chitosan, such as N-carboxymethyl chitosan or N-carboxybutyl chitosan, for instance the product sold under the name Chitoglycan by the company Sinerga SPA (INCI name Carboxymethyl chitosan);

(8) amphoteric polymers of the -D-X-D-X type chosen from:
a) polymers obtained by the action of chloroacetic acid or sodium chloroacetate on compounds comprising at least one unit of formula (X):

-D-X-D-X-D- (X)
where D denotes a group of formula (XI)

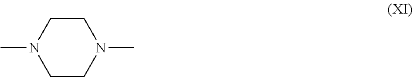

and X denotes the symbol E or E′, E or E′, which may be identical or different, denote a divalent group that is an alkylene group with a straight or branched chain containing up to 7 carbon atoms in the main chain, which is unsubstituted or substituted with hydroxyl groups and which can comprise, in addition to the oxygen, nitrogen and sulfur atoms, 1 to 3 aromatic and/or heterocyclic rings; the oxygen, nitrogen and sulfur atoms being present in the form of ether, thioether, sulfoxide, sulfone, sulfonium, alkylamine or alkenylamine groups, hydroxyl, benzylamine, amine oxide, quaternary ammonium, amide, imide, alcohol, ester and/or urethane groups;
b) polymers of formula (XII):

-D-X-D-X— (XII)
where D denotes a group of formula (XIII)

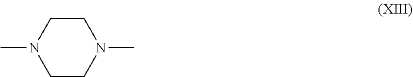

and X denotes the symbol E or E′ and at least once E′; E having the meaning given above and E′ is a divalent group that is an alkylene group with a straight or branched chain having up to 7 carbon atoms in the main chain, which is unsubstituted or substituted with one or more hydroxyl groups and containing one or more nitrogen atoms, the nitrogen atom being substituted with an alkyl chain that is optionally interrupted by an oxygen atom and necessarily comprising one or more carboxyl functions or one or more hydroxyl functions and betainized by reaction with chloroacetic acid or sodium chloroacetate;

(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkylaminoalkanol. These copolymers can also comprise other vinyl comonomers such as vinylcaprolactam.

Among the amphoteric film forming polymers mentioned above that are most particularly preferred according to the invention, mention will be made of those of family (3), such as the copolymers of which the INCI name is Octylacrylamide/acrylates/butylaminoethyl methacrylate copolymer, such as the products sold under the names Amphomer®, Amphomer® LV 71 or Balance® 47 by the company Akzo Nobel and those of family (4), such as the methyl methacrylate/methyl dimethylcarboxymethyl-ammonioethyl methacrylate copolymers sold, for example, under the name Diaformer Z-301N or Z-301W by the company Clariant.

Non-ionic film forming polymers that may be used according to the present invention may be chosen, for example, from:
polyalkyloxazolines;
vinyl acetate homopolymers;
vinyl acetate copolymers, for instance copolymers of vinyl acetate and of acrylic ester;
copolymers of vinyl acetate and of ethylene, or copolymers of vinyl acetate and of maleic ester, for example of dibutyl maleate;
homopolymers and copolymers of acrylic esters, for instance copolymers of alkyl acrylates and of alkyl methacrylates, such as the products provided by the company Rohm GmbH under the name Eudragit® NE 30 D (INCI name Acrylates copolymer);
copolymers of acrylonitrile and of a non-ionic monomer, chosen, for example, from butadiene and alkyl (meth)acrylates;
styrene homopolymers;
styrene copolymers, for instance copolymers of styrene, of alkyl acrylate and of alkyl methacrylate; copolymers of styrene and of butadiene, or copolymers of styrene, of butadiene and of vinylpyridine;
polyamides;
vinyllactam homopolymers, such as the vinylpyrrolidone homopolymers sold, for example, under the names Luviskol® K30 powder by the company BASF or PVP K30L or K60 solution or K90 by the company ISP, or such as the polyvinylcaprolactam sold under the name Luviskol® Plus by the company BASF (INCI name PVP);
vinyllactam copolymers, such as a poly(vinylpyrrolidone/vinyllactam) copolymer sold under the trade name Luvitec® VPC 55K65W by the company BASF, poly(vinylpyrrolidone/vinyl acetate) copolymers, such as those sold under the name PVP/VA® S630L, E735, E635 and W735 by the company ISP, Luviskol® VA 73, VA 64 and VA 37 by the company BASF (INCI name VP/VA copolymer); and vinylpyrrolidone/methacrylamide/vinylimidazole terpolymers, for instance the product sold under the name Luviset® Clear by the company BASF (INCI name VP/methacrylamide/vinyl imidazole copolymer).

The alkyl groups of the abovementioned non-ionic polymers may have from 1 to 6 carbon atoms.

In certain exemplary embodiments, it is also possible to use film forming polymers of grafted silicone type comprising a polysiloxane portion and a portion constituted of a non-silicone organic chain, one of the two portions constituting the main chain of the polymer and the other being grafted to the main chain.

These polymers are described, for example, in patent applications EP-A-0 412 704, EP-A-0 412 707, EP-A-0 640 105 and WO 95/00578, EP-A-0 582 152 and WO 93/23009 and patents U.S. Pat. Nos. 4,693,935, 4,728,571 and 4,972,037.

These polymers may be amphoteric, anionic or non-ionic.

Such polymers are, for example, copolymers that may be obtained by free radical polymerization from the monomer mixture formed:

a) of 50% to 90% by weight of tert-butyl acrylate,

b) of 0% to 40% by weight of acrylic acid,

c) of 5% to 40% by weight of a silicone macromer of formula (XIX):

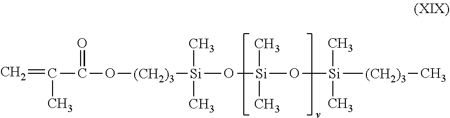

in which v is a number ranging from 5 to 700, the weight percentages being calculated relative to the total weight of the monomers.

Other examples of grafted silicone polymers include polydimethylsiloxanes (PDMSs) to which mixed polymer units of the poly(meth)acrylic acid type and of the poly(alkyl (meth)acrylate) type are grafted via a thiopropylene-type connecting link and polydimethylsiloxanes (PDMSs) to which polymer units of the poly(isobutyl (meth)acrylate) type are grafted via a thiopropylene-type connecting link.

Grafted silicone polymers are, for example, sold under the names Silicone Plus Polymer® VS80 and VA70 by 3M (INCI names Polysilicone-8 and Polysilicone-7 respectively).

Another type of silicone film forming polymer that may be mentioned is the product Luviflex® Silk sold by BASF (INCI name PEG/PPG-25/25 dimethicone/acrylates Copolymer).

As film forming polymers, it is also possible to use functionalized or non-functionalized, cationic, non-ionic, anionic or amphoteric, silicone or non-silicone polyurethanes, or mixtures thereof.

The polyurethanes that may be used in the disclosed compositions are those described in patent applications EP 0 751 162, EP 0 637 600, EP 0 648 485 and FR 2 743 297, and patent applications EP 0 656 021 and WO 94/03510 from the company BASF and EP 0 619 111 from the company National Starch.

Mention may be made, as polyurethanes suitable in the present invention, of the products sold under the names Luviset PUR® and Luviset® Si PUR by the company BASF (INCI names Polyurethane-1 and Polyurethane-6 respectively).

As yet further non-limiting examples, polymers useful according to the disclosure may be chosen from polysaccharides. Non-limiting examples of polysaccarides include oxidized inulins, celluloses, starches, guar gums, xanthan gums, pullulan gums, alginate gums, agar-agar gums, carrageenan gums, gellan gums, chitosan, gums arabic, xyloses and tragacanth gums, and derivatives thereof, cellobiose, maltodextrin, scleroglucan, chitosan, ulvan, fucoidan, alginate, pectin, heparin and hyaluronic acid, or mixtures thereof.

In certain exemplary embodiments, polysaccharides useful according to the disclosure may be chosen from gums. Non-limiting examples of gums include acacia, agar, algin, alginic acid, ammonium alginate, amylopectin, calcium alginate, calcium carrageenan, carrageenan, dextrin, gelatin, gellan gum, guar gum, gum Arabic, hydroxypropyl guar, guar hydroxypropyltrimonium chloride, hydroxypropyl guar hydroxypropyltrimonium chloride, karaya gum, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof.

Further non-limiting examples of polymers useful according to the disclosure include non-cellulose cationic polysaccharides, such as guar gums containing trialkylammonium cationic groups. Suitable cationic guar gum derivatives include those given the PCPC (Personal Care Products Council, formerly CTFA, designation) of guar hydroxypropyl trimonium chloride, available commercially for example as JAGUAR C135. Other suitable materials include that known as JAGUAR C15, JAGUAR C17, and JAGUAR C16 which is a hydroxypropylated cationic guar derivative containing a low level of substituent groups as well as cationic quaternary ammonium groups. Guar hydroxypropyl trimonium chloride, may also be available commercially for example as N-HANCE CG13 from the company Ashland. Also suitable is hydroxypropyl guar hydroxypropyltrimonium chloride, commercially available as JAGUAR 162.

In an exemplary embodiment, the at least one film forming polymer may be chosen from acrylate-based polymers, polyurethanes, and polysaccharides or mixtures thereof. In a further exemplary embodiment, the polymer may be chosen from PVM/MA (Poly(methyl vinyl ether-alt-maleic acid) (e.g., sold under the name Gantrez®), carrageenan, gum Arabic, oxidized inulin, alginate, xanthan gum, xylan, chitosan, or mixtures thereof. In yet a further exemplary embodiment, the polymer may be PVM/MA (Poly(methyl vinyl ether-alt-maleic acid) (e.g., sold under the name Gantrez®), carrageenan, or mixtures thereof.

The total amount of the at least one film forming polymer may vary, but typically ranges from about 0.001% to about 10%, such as from about 0.01% to about 5%, about 0.05% to about 3%, about 0.1% to about 2%, or about 0.5% to about 1%, based on the total weight of the composition. For example, the total amount of the film forming polymer may range from about 0.01% to about 10%, about 0.01% to about 9%, about 0.01% to about 8%, about 0.01% to about 7%, about 0.01% to about 6%, about 0.01% to about 5%, about 0.01% to about 4%, about 0.01% to about 3%, about 0.01% to about 2%, about 0.01% to about 1%, about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1%, about 0.5% to about 10%, about 0.5% to about 9%, about 0.5% to about 8%, about 0.5% to about 7%, about 0.5% to about 6%, about 0.5% to about 5%, about 0.5% to about 4%, about 0.5% to about 3%, about 0.5% to about 2%, about 0.5% to about 1.5%, or about 0.5% to about 1%, including all ranges and sub-ranges there between, based on the total weight of the composition. In certain embodiments, the at least one film forming polymer may be present in an amount of about 0.05%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, or about 2%, based on the total weight of the composition.

Optionally, the weight ratio of the total amount of polyphenol to the film-forming polymer may be chosen to range from about 1:100 to about 100:1, about 1:30 to about 30:1, such as about 1:10 to about 10:1, or about 1:4 to about 4:1, about 1:3 to about 3:1, or about 1:2 to about 2:1. In some embodiments, the weight ratio of the total amount of polyphenol to the film-forming polymer is greater than 1, for example ranges from about 1 to about 50, about 1 to about 25, about 1 to about 10, or about 5 to about 10. For example, the weight ratio of the total amount of polyphenol to the film-forming polymer may be about 5, about 10, about 25, or about 50.

In at least certain embodiments, the compositions are free or substantially free of film forming polymers.

In some embodiments, the compositions are free or substantially free of synthetic gums, silicones, and/or synthetic polymers. For example, the compositions may include less than about 3%, less than about 2%, less than about 1%, or less than about 0.5% of synthetic gums, silicones, and/or synthetic polymers, and in some embodiments comprise no synthetic gums, silicones, and/or synthetic polymers. In some embodiments, however, the compositions may comprise synthetic gums, silicones, and/or synthetic polymers. Non-limiting examples of silicones include amine-functionalized silicones (e.g., amodimethicone), dimethicone, bis-aminopropyl dimethicone, trimethyl silylamodimethicone, etc.

pH Adjusters

Compositions according to the disclosure may optionally also contain acid and alkali pH adjusters. Such pH adjusters include, but are not limited to, sodium metasilicate, silicate compounds, citric acid, ascorbic acid, and carbonate compounds. The disclosed compositions may also be substantially free of acid and alkali pH adjusters.

It may, in at least certain embodiments, be beneficial to adjust the pH of the compositions in order to either increase or decrease styling hold. For example, the pH of the composition may be adjusted such that it is approximately at or below the pKa of the polyphenol, or approximately at or below the first pKa of the case of polyphenols having more than one pKa. Without wishing to be bound by theory, adjusting the pH of the composition to be approximately at or below the (first) pKa of the polyphenol may aid in crosslinking of the polymer by the polyphenol. By way of example only, in an embodiment where tannic acid is used, the pH of the disclosed compositions may advantageously be at or below about 6, such as in the range of about 3 to about 8, about 3 to about 7.5, about 3 to about 7, about 3 to about 6.5, about 3 to about 6, about 3 to about 5.5, about 3 to about 5, about 4 to about 8, about 4 to about 7.5, about 4 to about 7, about 4 to about 6.5, about 4 to about 6, about 4 to about 5.5, about 4 to about 5, about 5 to about 8, about 5 to about 7.5, about 5 to about 7, about 5 to about 6.5, or about 5 to about 6.

Solvents

In certain exemplary embodiments, compositions according to the disclosure comprise at least one physiologically acceptable medium. The physiologically acceptable medium may be chosen from water or a mixture of water and at least one cosmetically acceptable solvent. Non-limiting examples of cosmetically acceptable solvents include C2-C4 lower alcohols, such as ethanol and isopropanol; polyols, especially those containing from 2 to 6 carbon atoms, for instance glycerol, propylene glycol, butylene glycol, pentylene glycol, hexylene glycol, dipropylene glycol or diethylene glycol; polyol ethers, for instance 2-butoxyethanol, propylene glycol monomethyl ether and diethylene glycol monomethyl ether or monoethyl ether; and mixtures thereof.

In one exemplary embodiment, the composition comprises a solvent or solvent mixture in an amount up to about 99.9%, such as ranging from about 50% to about 99%, about 55% to about 95%, or about 60% to about 90%, by weight relative to the weight of the composition. For example, the composition may comprise water in an amount up to about 99%, such as, for example, an amount ranging from about 50 to about 95%, or from about 60% to about 90%, by weight relative to the weight of the composition.

Additional Components

The compositions according to the disclosure may optionally also comprise additives useful or desired for preparing hair styling compositions. For example, it may be beneficial to include components that will aid in application of the composition, such as glycerol. Exemplary and non-limiting additives include nacreous agents, dyes or pigments, fragrances, mineral, plant or synthetic oils, waxes, vitamins, proteins including ceramides, vitamins, UV-screening agents, free-radical scavengers, antidandruff agents, hair-loss counteractants, hair restorers, preserving agents, and mixtures thereof. A person skilled in the art will take care to select the optional additives and the amount thereof such that they do not harm the properties of the compositions of the present disclosure.

The compositions of certain embodiments may comprise stabilizers, for example sodium chloride, magnesium dichloride or magnesium sulfate.

The compositions according to the disclosure may additionally comprise cosmetic adjuvants chosen from fragrances, pigments, chelating agents, softeners, antioxidants, opacifiers, stabilizers, moisturizing agents, vitamins, bactericides, preservatives, polymers, thickening agents, or any other ingredient commonly used in cosmetics for this type of application.

In certain embodiments, these additives are generally present in an amount ranging up to about 40% by weight of active material relative to the total weight of the composition, such as up to about 30%, up to about 20%, up to about 15%, up to about 10%, up to about 5%, such as from 0.01% to 30%.

If desired, a person of skill in the art can select the additives or amounts thereof in order to maintain the desired properties of the compositions.

In certain embodiments, at least 75%, such as at least 80%, at least 85%, at least 90%, or at least 95% of the disclosed materials used in the compositions are plant-based. In one exemplary embodiment, compositions according to the disclosure demonstrate desirable cosmetic properties while comprising mostly, such as at least about 95%, natural origin ingredients.

The compositions described herein may be in any suitable physical form. Suitable forms include, but are not limited to, low to moderate viscosity liquids, lotions, milks, gel creams, creams, pastes, clays, conditioners, masks, and the like.

The compositions may be packaged in a variety of different containers, such as, for example, a ready-to-use container. Non-limiting examples of useful packaging include tubes, jars, caps, unit dose packages, and bottles, including squeezable tubes and bottles, bottles configured with pump dispensers, and spray bottles, including pumper spray bottles.

### II. Methods

Methods according to the disclosure comprise applying compositions described herein to the hair, and styling the hair. In one exemplary embodiment, the composition is a leave-in hair styling composition. Methods of treating hair according to the disclosure may include applying a composition according to disclosure to hair (wet, damp, or dry hair). The composition may be applied to the hair before, during or after any styling process, such as a process for curling (e.g., using curlers) or straightening the hair. The methods may involve elongating the curl of the hair. Elongation involves any method wherein a curl is allowed to flow away from the scalp, as opposed to maintaining a tight curl against the scalp. The composition may be applied to the hair before, with, or after other hair compositions (e.g., a shampoo, a conditioner, a mask, a cream, a lotion, a gel, a spray, etc.). Although the use of heat is not required to achieve hair styling using compositions according to the disclosure, an individual may choose to heat the styled hair before, during, or after the composition is applied to the hair, such as, for example, using a hair dryer.

It is to be understood that although compositions and/or hair styling processes according to the disclosure generally demonstrate one or more of the properties described herein, compositions according to the disclosure may not demonstrate all or some of the disclosed properties, yet the compositions and methods of styling hair are still within the scope of the disclosure.

The following definitions are provided for the present disclosure only.

As used herein, the term “hair” is meant to include only hair on the scalp of the head, and does not include eyelashes or eyebrows.

The terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

The terms “a,” “an,” “the,” and “at least one” are understood to encompass the plural as well as the singular.

Thus, the term “a mixture thereof” also relates to “mixtures thereof.” Throughout the disclosure, if the term “a mixture thereof” is used, following a list of elements as shown in the following example where letters A-F represent the elements: “one or more elements selected from the group consisting of A, B, C, D, E, F, or mixtures thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting.

The expression “one or more” means “at least one” and thus includes individual components as well as mixtures/combinations.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

Unless otherwise indicated, all percentages herein are by weight, relative to the weight of the total composition.

The term “substantially free” or “essentially free” as used herein means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure.

The term “substantially free” or “essentially free” as used herein may also mean that the specific material is not added to the composition but may still be present in a raw material that is included in the composition.

The term “synthetic” means a material that is not of natural origin. The term “natural” means a material of natural origin, such as derived from plants, which also cannot be subsequently chemically or physically modified.

The term “organic” means a material that is produced substantially without or essentially without the use of synthetic materials. The term “substantially without” or “essentially without” as used herein means the specific material may be used in a manufacturing process in small amounts that do not materially affect the basic and novel characteristics of the compositions according to the disclosure. The term “substantially without” or “essentially without” as used herein may also mean that the specific material is not used in a manufacturing process but may still be present in a raw material that is included in the composition.

“Cosmetic composition” encompasses many types of compositions for application to hair, for example, hair lotions, hair creams, hair gel creams, hair conditioners, hair masques (masks), etc. In an embodiment, the compositions are leave-on hair compositions.

“Hair styling compositions” are compositions that, when applied to the hair, allow the hair to achieve and/or retain a desired shape.

It will be apparent to those skilled in the art that various modifications and variations can be made in the compositions and methods according to the disclosure without departing from the spirit or scope of the disclosure. Thus, it is intended that the disclosure cover such modifications and variations and their equivalents.

### EXAMPLES

The following examples are intended to be non-limiting and explanatory in nature only.

### Example 1

The following hair styling compositions were prepared by mixing the components, as disclosed in Table 1. Amounts are expressed in wt % of active materials:

TABLE 1

| | Composition | | | | | |
|---|---|---|---|---|---|---|
| | 1a | 1b | 1c | 1d | 1e | 1f |
| Gantrez ® | | | | | | |
| PVM/MA | | | | | | |
| Copolymer | | | | | | |
| (CAS | | | | | | |
| Number | | | | | | |
| 25153-40-6) | — | — | 0.5% | 0.5% | 0.5% | 0.5% |
| Tannic acid | — | 5.0% | — | 0.5% | 1.0% | 5.0% |
| Water | Q.S. 100 | Q.S. 100 | Q.S. 100 | Q.S. 100 | Q.S. 100 | Q.S. 100 |

Each composition was applied to a separate swatch of straight, wet, natural, Caucasian hair at 0.4 grams per gram of hair and worked through the hair with fingers and a comb. Then, 0.4 g/g of 0.002% citric acid was applied to the swatches that were coated with compositions 1a, 1d, 1e, and 1f. The hair swatches were then wrapped around hair rollers and air-dried overnight. The hair swatches were then released from rollers and hung vertically.

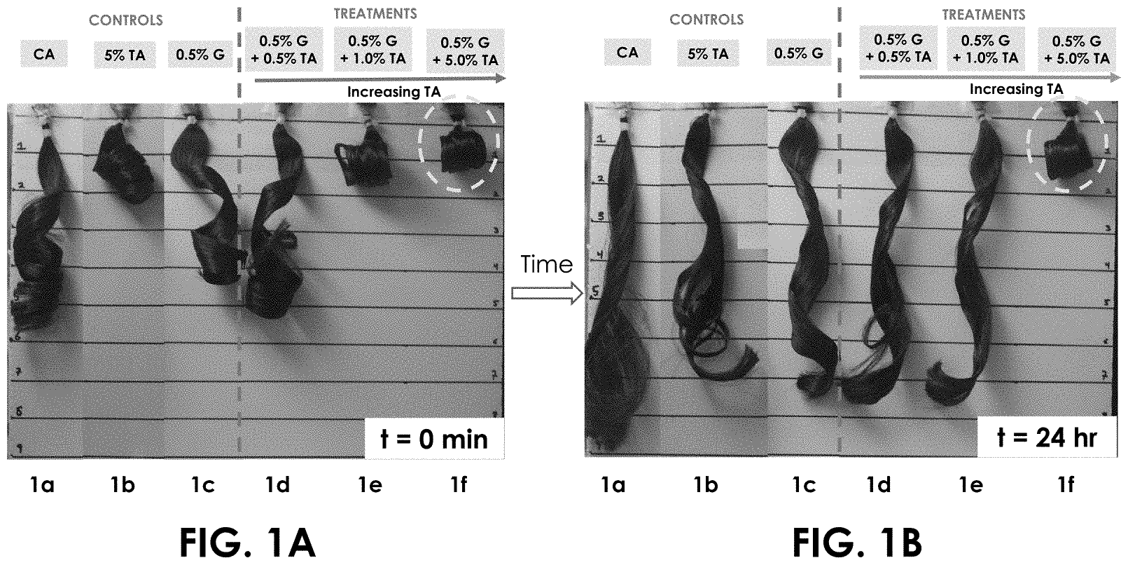

Thus, at the initial treatment, time=zero (FIG. 1A), the hair treated with 1a to 1f compositions (plus citric acid for 1a, 1d, 1e, and 1f) became curly to varying degrees after using rollers on the hair, with 1b, 1e, and 1f treatments providing the tightest or highest degree of curl. After exposing that hair to high humidity, the treatment of the hair with the 1f composition comprising the Gantrez® PVM/MA Copolymer and tannic acid followed by citric acid surprisingly demonstrated unexpected synergistic beneficial styling properties, including improving the effects of curl retention and imparting high humidity style retention to hair (no apparent change in degree of curliness between time=zero and after exposure to high humidity). The hair treated with 1b, 1c, 1d, and 1e compositions still retained some degree of curliness compared to time=zero results but the hair treated with 1b composition appeared to be curlier than hair treated with 1c, 1 d, and 1e compositions.

### Example 2

The following hair styling compositions were prepared by mixing the components, as disclosed in Table 2. Amounts are expressed in wt % of active materials:

TABLE 2

| | Composition | |
|---|---|---|
| | 2a | 2b |
| Gantrez ® PVM/MA Copolymer | 0.5% | 0.5% |
| (CAS Number 25153-40-6) | | |
| Tannic acid | — | 5.0% |
| Water | Q.S. 100 | Q.S. 100 |

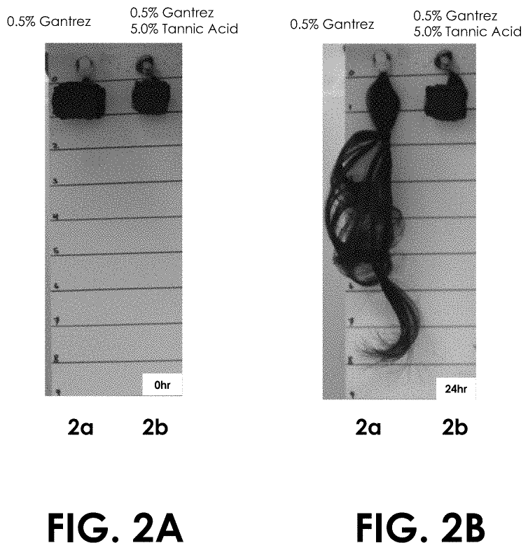

Compositions 2a and 2b were each applied to a separate swatch of straight, wet, natural, Caucasian hair at 0.15 grams per gram of hair and worked through the hair with fingers and a comb. The hair was then wrapped around a curler and air-dried overnight. The hair was then released from curlers, hung vertically, and determined to be curly. (FIG. 2A). The hair remained hung for 24 hours at 80% relative humidity and 25° C. After 24 hours, the swatches were determined to still be curly (composition 2b) or the curls elongated (composition 2a). (FIG. 2B).

The composition comprising the Gantrez® PVM/MA Copolymer and tannic acid (compositions 2b) surprisingly demonstrated unexpected synergistic beneficial styling properties, including improving the effects of curl retention and imparting high humidity style retention to hair.

### Example 3

The following hair styling compositions were prepared by mixing the components, as disclosed in Table 3. Amounts are expressed in wt % of active materials:

TABLE 3

| | Composition | |
|---|---|---|
| | 3a | 3b |
| Carrageenan | 0.2% | 0.2% |
| Tannic acid | — | 2.0% |
| Water | Q.S. 100 | Q.S. 100 |

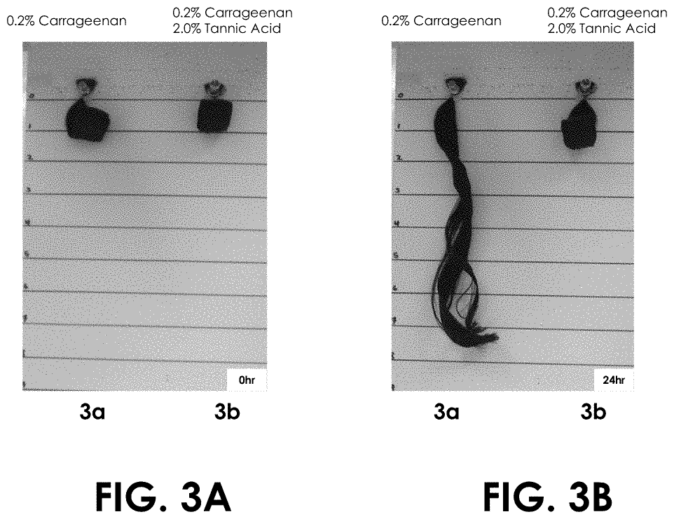

Each composition was applied to separate swatches of straight, wet, natural, Caucasian hair at 0.15 grams per gram of hair and worked through the hair with fingers and a comb. Each swatch of hair was then wrapped around a curler and air-dried overnight. The hair was then released from curlers, hung vertically, and all swatches were determined to be curly (FIG. 3A). The hair remained hung for 24 hours at 80% relative humidity and 25° C. After 24 hours, the hair achieved and retained its curl (composition 3b) or the curls elongated (composition 3a)(FIG. 3B).

The composition comprising carrageenan and tannic acid (compositions 3b) surprisingly demonstrated unexpected synergistic beneficial styling properties, including improving the effects of curl retention and imparting high humidity style retention to hair.

### Example 4

The following hair styling compositions were prepared by mixing the components, as disclosed in Table 4. Amounts are expressed in wt % of active materials:

TABLE 4

| | Composition | |
|---|---|---|
| | 4a | 4b |
| Alginate | 0.2% | 0.2% |
| Tannic acid | — | 2.0% |
| Water | Q.S. 100 | Q.S. 100 |

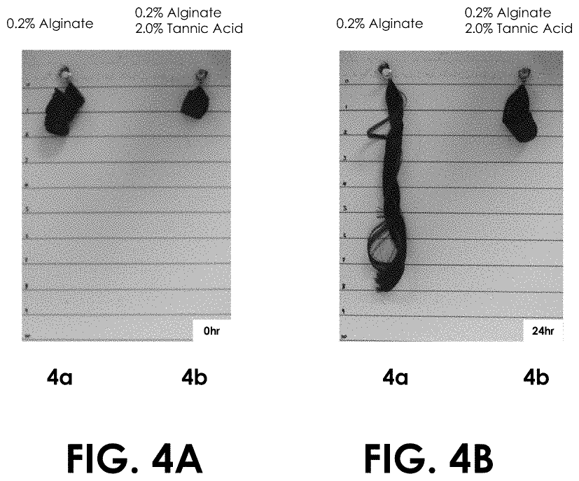

Each composition was applied to separate swatches of straight, wet, natural, Caucasian hair at 0.15 grams per gram of hair and worked through the hair with fingers and a comb. Each swatch of hair was then wrapped around a curler and air-dried overnight. The hair was then released from curlers, hung vertically, and all swatches were determined to be curly (FIG. 4A). The hair remained hung for 24 hours at 80% relative humidity and 25° C. After 24 hours, the hair achieved and retained its curl (composition 4b) or the curls elongated (composition 4a)(FIG. 4B).

The composition comprising alginate and tannic acid (composition 4b) surprisingly demonstrated unexpected synergistic beneficial styling properties, including improving the effects of curl retention and imparting high humidity style retention to hair.

### Example 5

The following hair styling compositions were prepared by mixing the components, as disclosed in Table 5. Amounts are expressed in wt % of active materials:

TABLE 5

| | Composition | |
|---|---|---|
| | 5a | 5b |
| Xanthan Gum | 0.5% | 0.5% |
| Tannic acid | — | 5.0% |
| Water | Q.S. 100 | Q.S. 100 |

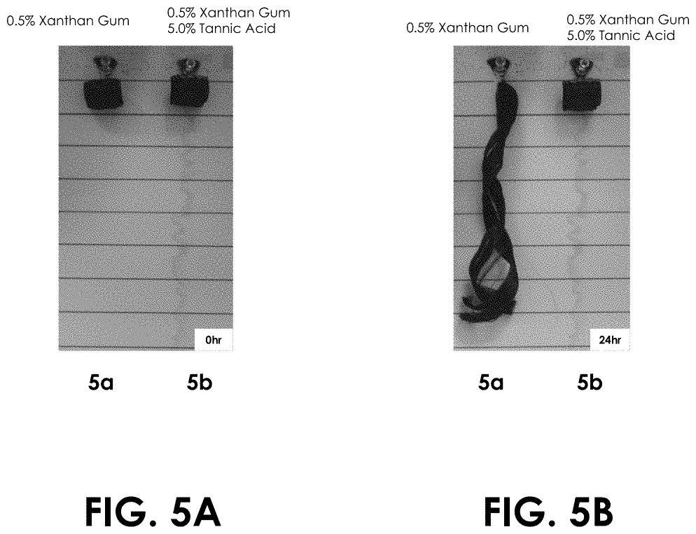

Each composition was applied to separate swatches of straight, wet, natural, Caucasian hair at 0.15 grams per gram of hair and worked through the hair with fingers and a comb. Each swatch of hair was then wrapped around a curler and air-dried overnight. The hair was then released from curlers, hung vertically, and all swatches were determined to be curly (FIG. 5A). The hair remained hung for 24 hours at 80% relative humidity and 25° C. After 24 hours, the hair achieved and retained its curl (composition 5b) or the curls elongated (composition 5a) (FIG. 5B).

The composition comprising xanthan gum and tannic acid (composition 5b) surprisingly demonstrated unexpected synergistic beneficial styling properties, including improving the effects of curl retention and imparting high humidity style retention to hair.

### Example 6

The following hair styling compositions were prepared by mixing the components, as disclosed in Table 6. Amounts are expressed in wt % of active materials:

TABLE 6

| | Composition | |
|---|---|---|
| | 6a | 6b |
| Gum | 1.0% | 1.0% |
| Arabic | | |
| Tannic acid | — | 5.0% |
| Water | Q.S. 100 | Q.S. 100 |

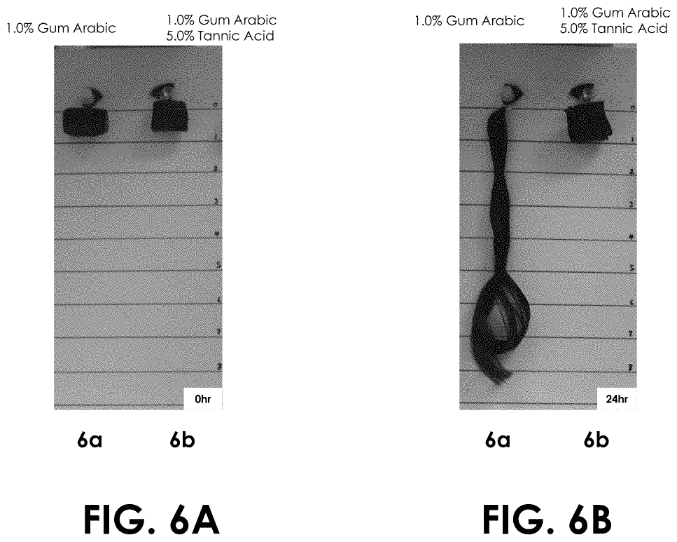

Each composition was applied to separate swatches of straight, wet, natural, Caucasian hair at 0.15 grams per gram of hair and worked through the hair with fingers and a comb. Each swatch of hair was then wrapped around a curler and air-dried overnight. The hair was then released from curlers, hung vertically, and all swatches were determined to be curly (FIG. 6A). The hair remained hung for 24 hours at 80% relative humidity and 25° C. After 24 hours, the hair achieved and retained its curl (composition 6b) or the curls elongated (composition 6a) (FIG. 6B).

The composition comprising gum Arabic and tannic acid (composition 6b) surprisingly demonstrated unexpected synergistic beneficial styling properties, including improving the effects of curl retention and imparting high humidity style retention to hair.

### Example 7

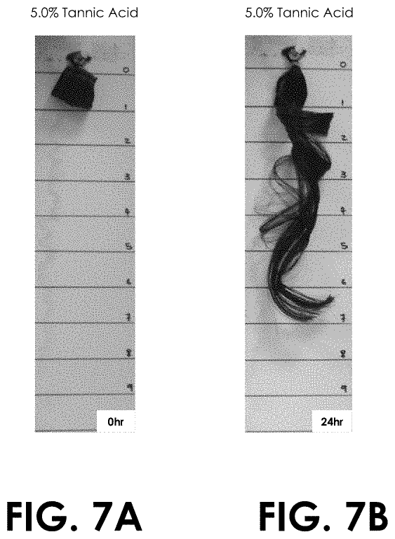

A hair styling composition with 5.0% tannic acid in water was prepared (expressed in wt % of active materials). The composition was applied to a swatch of straight, wet, natural, Caucasian hair at 0.15 grams per gram of hair and worked through the hair with fingers and a comb. The swatch of hair was then wrapped around a curler and air-dried overnight. The hair was then released from curlers, hung vertically, and all swatches were determined to be curly (FIG. 7A). The hair remained hung for 24 hours at 80% relative humidity and 25° C. After 24 hours, the curls elongated (FIG. 7B).

The composition comprising tannic acid surprisingly demonstrated that it has the ability to curl or alter the shape of hair on its own.

### Example 8

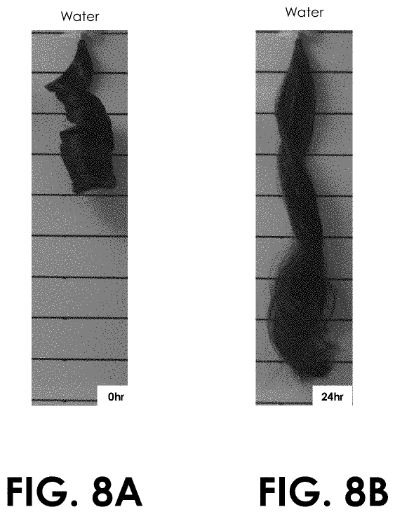

Water was applied to a swatch of straight, wet, natural, Caucasian hair at 0.15 grams per gram of hair and worked through the hair with fingers and a comb. The swatch of hair was then wrapped around a curler and air-dried overnight. The hair was then released from curlers, hung vertically, and all swatches were determined to be curly (FIG. 8A). The hair remained hung for 24 hours at 80% relative humidity and 25° C. After 24 hours, the hair had a slight wave. (FIG. 8B).

### Example 9

The following hair styling compositions were prepared by mixing the components, as disclosed in Table 7. Amounts are expressed in wt % of active materials:

TABLE 7

| | Composition | |
|---|---|---|
| | 7a | 7b |
| Gantrez ® PVM/MA Copolymer | 0.5% | — |
| (CAS Number 25153-40-6) | | |
| Carageenan | — | 0.5% |
| Glycerol | 1.0% | 1.0% |
| Tannic acid | 5.0% | 5.0% |
| Citric Acid | 0.1% | 0.1% |
| Water | Q.S. 100 | Q.S. 100 |

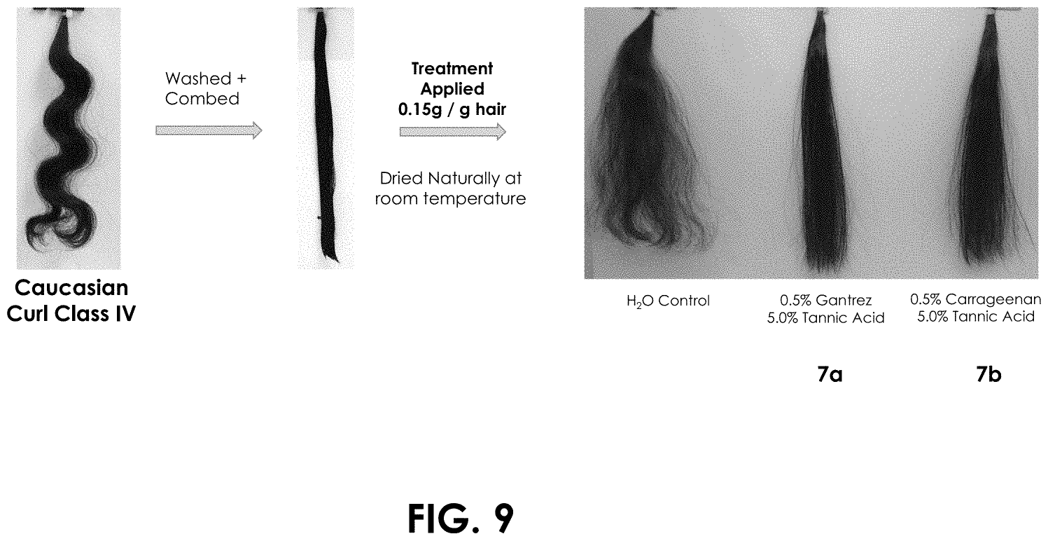

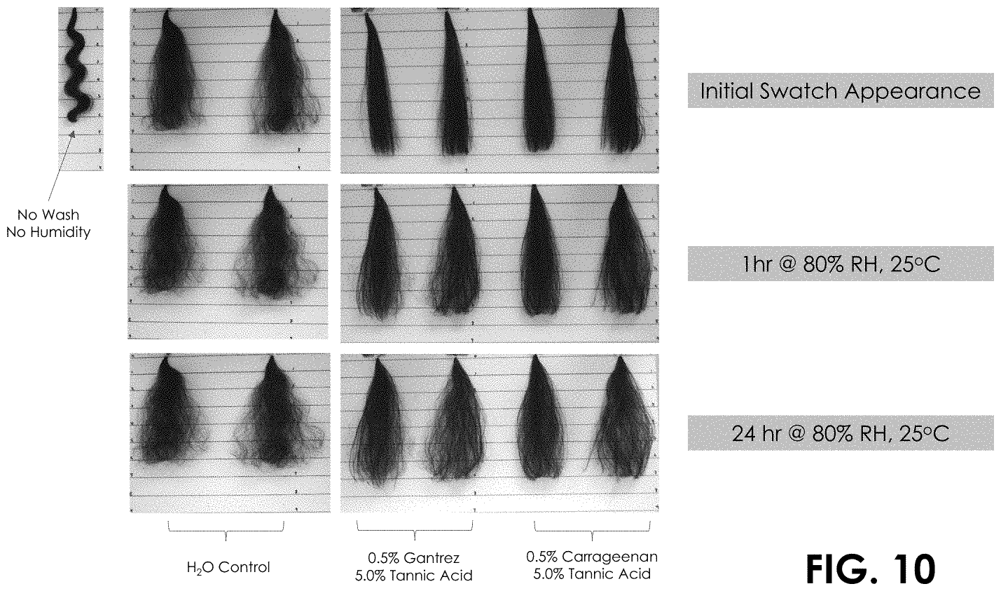

Swatches of curly, natural, Caucasian, hair was pre-washed with shampoo, and combed straight while wet. The compositions were applied to separate swatches of hair at 0.15 grams per gram of hair, and worked through the hair with fingers and three passes with a comb. The hair was then hung vertically, air-dried naturally at room temperature, and determined to be straight when dry (FIG. 9). The hair was then exposed to 80% relative humidity and 25° C. for 24 hours while hanging. After the first (1) hour and the full 24 hour period, the treated hair was still straight (FIG. 10).

The example demonstrates that compositions comprising the Gantrez® PVM/MA Copolymer and tannic acid, or carageenan and tannic acid, surprisingly provides unexpected synergistic styling properties, including improving the effects of hair straightening and imparting high humidity style retention to hair.

### Example 10

The following hair styling compositions were prepared by mixing the components, as disclosed in Table 8. Amounts are expressed in wt % of active materials:

TABLE 8

| | Composition |
|---|---|
| | 8a |
| Carageenan | 0.5% |
| Tannic acid | 5.0% |
| Water | Q.S. 100 |

Swatches of natural, Caucasian curly hair (medium degree of curliness) were pre-washed with a conventional shampoo, combed straight with a wide-tooth comb to remove knots and tangles, and cut to 7.5 inches in length per swatch. Composition 8a or the control (water) was applied to separate swatches of hair at 0.15 grams per gram of hair, and worked through the hair with fingers and five passes with a wide-toothed comb and five passes of a fine-toothed comb. Swatches were dried overnight at ambient conditions. Then the swatches were combed 10 times with a wide tooth comb prior to measurement.

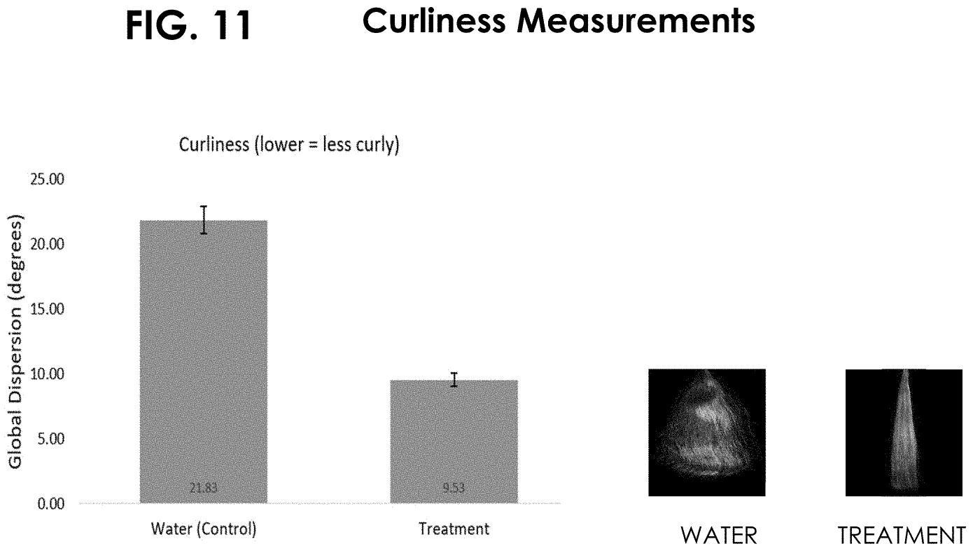

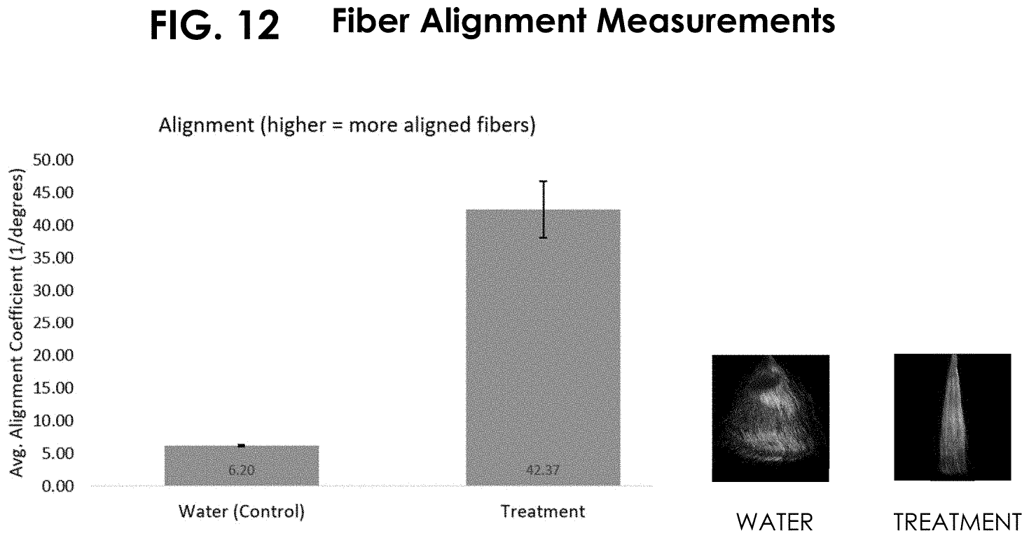

Degree of curliness and fiber alignment measurements were conducted on the hair fibers in the treated swatches using a technique involving polarization imaging and image analysis (based on the methods described in the article N. Lechocinski and S. Breugnot, S. Fiber Orientation Measurement Using Polarization Imaging, J. Cosmet. Sci., 62, 85-100 (2011)). The results are expressed in table 9 below by the parameters Global Dispersion (degrees) (see FIG. 11) and Average Alignment Coefficient (1/degrees or 1/°) (see FIG. 12).

TABLE 9

| | Water | Composition 8a |
|---|---|---|
| Global Dispersion | 21.83 | 9.53 |
| (degrees) | | |
| Ave. Alignment Coefficient | 6.2 | 42.37 |
| (1/°) | | |

A lower global dispersion value indicates that the degree of curliness of the hair is lower (i.e., the hair is straighter and more stretched out). A higher average alignment coefficient value indicates that the hair fibers are more aligned. Thus, the hair was straighter and more stretched out, and the fibers were more aligned are along the length of the swatch after treating the hair with composition 8a (“Treatment”) as compared to the hair treated with the control (water). This is also visually evident from the photograph of the hair swatches in FIGS. 11 and 12.

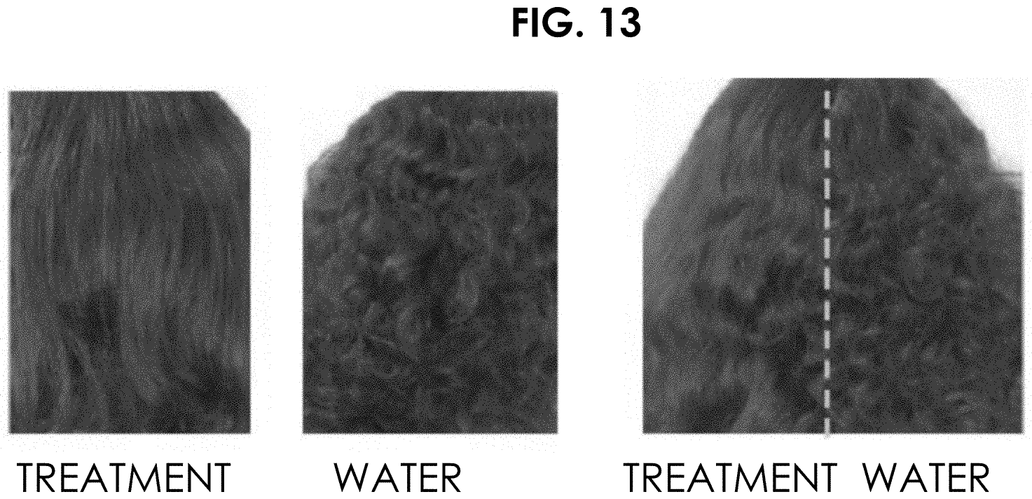

Composition 8a was also applied onto the hair of human volunteers in a half head study where one side of the head was treated with composition 8a and the other side was treated with water (the control). Composition 8a was applied to half of a head of each volunteer's head hair at in an amount sufficient to coat the hair fibers of each volunteer, and hair was then combed straight. Water was similarly applied to the other half of a head of each volunteer's head hair and combed straight. The hair was dried using a roller-ball dryer, and was then combed. The images in FIG. 13 are representative images from one volunteer in the half head study. It was visually observed that the curl pattern or degree of curliness of hair treated with composition 8a was significantly reduced or less along the length of the hair compared to the curl pattern or degree of curliness of the hair treated with water only (see FIG. 13). The hair treated with water retained the same natural curl pattern it had before it was treated with water.

1. A hair styling composition comprising:
(a) at least one polyphenol, and
(b) at least one film forming polymer,
wherein the pH of the composition is approximately at or below the pKa of the polyphenol.

2. The hair styling composition of claim 1, wherein the at least one film forming polymer is present in an amount ranging from about 0.01% to about 10%, based on the total weight of the composition.

3. The hair styling composition of claim 1, wherein the at least one film forming polymer is chosen from acrylate-based polymers, polyurethanes, polysaccharides, or mixtures thereof.

4. The hair styling composition of claim 1, wherein the at least one film forming polymer is chosen from PVM/MA Copolymer, carrageenan, gum Arabic, xanthan gum, alginate, or mixtures thereof.

5. The hair styling composition of claim 1, wherein the at least one polyphenol is present in an amount ranging from about 0.001% to about 20%, based on the total weight of the composition.

6. The hair styling composition of claim 1, wherein the at least one polyphenol is tannic acid.

7. The hair styling composition of claim 1, wherein the pH of the composition is less than about 8.

8. The hair styling composition of claim 1, wherein the weight ratio of the total amount of the polyphenol to the total amount of film forming polymer ranges from about 1:30 to about 30:1.

9. The hair styling composition of claim 1, wherein:
(a) the at least one polyphenol is tannic acid,
(b) the at least one film forming polymer is chosen from PVM/MA Copolymer, carrageenan, gum Arabic, xanthan gum, alginate, or mixtures thereof, and
(c) at least one solvent, and
wherein the pH of the composition is at or below 6.

10. The hair styling composition according to claim 9, wherein the tannic acid is present in an amount ranging from about 4% to about 6%, by weight relative to the total composition.

11. The hair styling composition according to claim 9, wherein the at least one film forming polymer is present in an amount ranging from about 0.05% to about 3%, by weight relative to the total composition.

12. The hair styling composition of claim 9, wherein the weight ratio of the total amount of the tannic acid to the total amount of film forming polymer ranges from about 5:1 to about 10:1.

13. The hair styling composition of claim 1, wherein the:
(a) the at least one polyphenol is tannic acid, and
(b) the at least one film forming polymer is chosen from a polymer of formula (I):
[CH₂CH(OCH₃)CH(CO₂H)CH(CO₂H)]n,
wherein n is a number from about 2,500 to about 17,500.

14. The hair styling composition of claim 1, wherein the composition comprises:
(a) from about 0.1% to about 10%, based on the total weight of the composition, of at least one polyphenol, wherein the at least one polyphenol is tannic acid, and
(b) from about 0.1% to about 2%, based on the total weight of the composition, of the at least one film forming polymer, wherein the at least one film forming polymer is chosen from a polymer of formula (I):
[CH₂CH(OCH₃)CH(CO₂H)CH(CO₂H)]n,
wherein n is a number from about 2,500 to about 17,500, and
wherein the pH of the composition is less than about 6.5.

15. The hair styling composition of claim 1, wherein:
(a) the at least one polyphenol is tannic acid, and
(b) the at least one film-forming polymer is chosen from carrageenan, alginate, xanthan gum, gum Arabic, or combinations thereof.

16. The hair styling composition of claim 1, wherein the composition comprises:
(a) from about 0.1% to about 10%, based on the total weight of the composition, of at least one polyphenol, wherein the at least one polyphenol is tannic acid, and
(b) from about 0.1% to about 2%, based on the total weight of the composition, of the at least one film forming polymer, wherein the film forming polymer is chosen from carrageenan, alginate, xanthan gum, gum Arabic, or combinations thereof,
wherein the pH of the composition is less than about 6.5.

17. A method for styling hair, said method comprising:
(1) applying to the hair a hair styling composition comprising:
(a) at least one polyphenol, and
(b) at least one film forming polymer,
wherein the pH of the composition is approximately at or below the pKa of the polyphenol; and
(2) styling the hair.

18. The method of claim 17, wherein the hair styling composition is applied to wet hair and the hair is dried after the hair is styled.

19. The method of claim 17, wherein styling the hair comprises straightening the hair.

20. The method of claim 17, wherein styling the hair comprises curling the hair.

21. The method of claim 17, wherein styling the hair comprises elongating the curl of the hair.

22. A hair styling composition comprising tannic acid.
